(19) United States
(12) Patent Application Publication
(10) Pub. No.: US 2026/0151586 A1
(43) Pub. Date: Jun. 4, 2026

(54) DEVICE FOR PERFORMING HYPOXIA TRAINING

(71) Applicant: Egor EGOROV, Berlin (DE)

(72) Inventor: Egor EGOROV, Berlin (DE)

(21) Appl. No.: 19/122,723

(22) PCT Filed: Oct. 19, 2023
(86) PCT No.: PCT/EP2023/079152

§ 371 Date: Apr. 18, 2025

(30) Foreign Application Priority Data
Oct. 19, 2022 (DE) 10 2022 127 597.3

(51) Int. Cl. A61M 16/20; B01D 53/14; B29L 30/00

(52) U.S. Cl. CPC A61M 16/22; A61M 16/12; A61M 16/208; A61M 2202/0225

(57) ABSTRACT

The invention relates to a device for providing a hypoxic gas mixture, said device having a gas reservoir, a gas delivery device, a supply line that is suitable and intended for conducting a gas mixture from the gas reservoir to the gas delivery device, and/or a discharge line that is suitable and intended for conducting a gas mixture from the gas delivery device to the gas reservoir, and a CO₂ absorber, characterized in that the CO₂ absorber is arranged in a carrier element. The invention further relates to a method for performing hypoxia training, comprising the method steps of inserting a container into a carrier element of a device for providing a hypoxic gas mixture, the container containing a CO₂ absorber, and connecting the gas delivery device to the mouth and/or nose of the user. The invention also relates to a container with a CO₂ absorber for performing hypoxia training, characterized in that the container is sealed.

[symbol]

The invention relates to a device for providing a hypoxic gas mixture, said device having a gas reservoir, a gas delivery device, a supply line that is suitable and intended to conduct a gas mixture from the gas reservoir to the gas delivery device, and/or a discharge line that is suitable and intended to conduct a gas mixture from the gas delivery device to the gas reservoir, and a CO₂ absorber, characterized in that the CO₂ absorber is arranged in a carrier element. The invention further relates to a method for performing hypoxia training, comprising the method steps of inserting a container into a carrier element of a device for providing a hypoxic gas mixture, the container containing a CO₂ absorber, and connecting the gas delivery device to the mouth and/or nose of the user. The invention also relates to a container with a CO₂ absorber for performing hypoxia training, characterized in that the container is sealed.

### PRIOR ART

Hypoxia can cause reactions in any cell in the body and can enable an increased energy metabolism. It can contribute to the activation of a variety of genes. Athletes, healthy people, and the sick may benefit from hypoxia.

It has been known for a long time that altitude training has beneficial effects. But how the slight lack of oxygen leads to an improvement in performance in the body could not be explained sufficiently until a few years ago. The observed increase in red blood cells was not sufficient to explain the changes in the body. The breakthrough for understanding came with the discovery of the hypoxia-inducible factor HIF-1-alpha. It provided the explanation for the comprehensive effect of altitude training. The abbreviation HIF stands for Hypoxia-Inducible Factor. Behind the technical term lies an oxygen sensor, which becomes active when there is not enough oxygen in the body cells. It controls one of the most important processes in the body for survival: the adaptation of cells, tissues and organs to a lack of oxygen. At the same time, it is a signal for self-repair in the body.

The best known positive effect of HIF is erythropoietin synthesis (EPO) in the kidney and liver. It served to explain the changes in the cardiovascular, respiratory and blood systems before the discovery of HIF. It is now clear that the performance improvement is much more comprehensive. The endothelial cells of the tunica intima react to the impact of hypoxia with an increased nitric oxide synthesis (NO). The gas has a decisive impact on the dilatation of the vessels. It leaves the endothelium and has a relaxing effect on smooth muscle cells in the surrounding tissue. On the intimal tunica itself, NO prevents platelet adhesion and aggregation. It is also interesting in this context that the endothelial cells form the angiogenetic factor VEGF under the impact of hypoxia. As a result of its formation, neoangiogenesis of the capillary vessels occurs in the context of hypoxia therapy. Very often, these additional vessels are located in damaged or poorly perfused tissue sections.

All known apparatuses for performing hypoxia training have at least one breathing reservoir into which, at the beginning of each diagnostic and therapeutic session, a certain amount of atmospheric air is introduced at the beginning of each diagnostic, training or therapy session. When the user connected to this reservoir inhales and exhales from it, the oxygen in this reservoir is consumed and an oxygen-deficient oxygen-containing gas mixture is created in this reservoir. This mixture is purified from excess carbon dioxide and the residual oxygen content is measured at least periodically during each session.

Patent specification WO 2012/005712 presents an apparatus for breathing with hypoxic gas mixtures, consisting of: a support frame; a respiratory reservoir with an inspiration valve, a first sensor of oxygen content and an absorber for CO₂ and H₂O; a controllable compressor connected to the respiratory reservoir via a puffing valve; a chamber for sampling and accumulation of aliquots of gas mixtures, equipped with a second sensor of oxygen content, a controllable adjustment valve and a controllable ejector; a user attachment; an inspiration line having a controllable center valve and a first flow meter for measuring the inspiratory rate; a T-fitting; a main expiration line equipped with a first air blower and connecting the attachment to the reservoir; an additional expiration line equipped with a second flow meter for measuring the volume of sample aliquots and a second air blower and connecting the connection to the chamber; additional sensor complex and control unit.

The device presented here is very complex in design and therefore very expensive to purchase. Its use is particularly complex for a private user and must therefore be carried out under professional supervision.

It is therefore an object of the present invention to provide a device for providing a hypoxic gas mixture which enables hypoxia training for a private user in an efficient, cost-effective and safe manner, even without professional supervision.

It is therefore a further object of the present invention to provide a method for performing hypoxia training, which enables safe and at the same time efficient performance of hypoxia training for a private user, even without professional supervision.

It is also an object of the invention to provide a container with a CO2 absorber for performing hypoxia training, which enables safe and at the same time efficient performance of hypoxia training for a private user, even without professional supervision.

It is therefore an object of the present invention to provide a device for providing a hypoxic gas mixture which enables hypoxia training for a private user in an efficient, cost-effective and safe manner, even without medical supervision.

Furthermore, it is an object of the present invention to provide a method for performing hypoxia training, which enables safe and at the same time efficient performance of hypoxia training for a private user, even without medical supervision.

It is also an object of the invention to provide a container with a CO2 absorber for performing hypoxia training, which enables safe and at the same time efficient performance of hypoxia training for a private user, even without medical supervision.

The stated object is achieved by means of the device for providing a hypoxic gas mixture according to claim 1. Further advantageous configurations of the invention are set out in the dependent claims.

The device according to the invention for providing a hypoxic gas mixture has a gas reservoir. The gas reservoir is an air reservoir that has the gas mixture for performing hypoxia treatment and/or hypoxia training. In addition, the device for providing a hypoxic gas mixture has a gas delivery device. The gas delivery device is usually embodied as a breathing mask that a user wears over the breathing openings (mouth and nose) to perform hypoxia treatment and/or hypoxia training. Furthermore, the device for providing a hypoxic gas mixture has a supply line that is suitable and intended to conduct a gas mixture from the gas reservoir to the gas delivery device and/or a discharge line that is suitable and intended to conduct a gas mixture from the gas delivery device to the gas reservoir. The supply and/or discharge line is usually embodied as a flexible hose and connects the gas delivery device to the gas reservoir in a gas-tight manner. In addition, the device for providing a hypoxic gas mixture has a carrier element in which a CO₂ absorber can be arranged according to the invention.

In a refinement of the invention, the CO₂ absorber can be placed in the carrier element and removed from it. Optionally, the carrier element is suitable and also intended to accommodate the CO₂ absorber as a so-called disposable.

A disposable within the meaning of the invention is a packaging unit of a CO₂ absorber, preferably with a fixed CO₂ absorber content. The CO₂ absorber can, for example, be packaged in a container itself or be in loose form. In packaged form, the disposable has an external shape determined by the carrier element of the device for providing a hypoxic gas mixture.

The device according to the invention is a so-called pendulum rebreather, which means that the same air is inhaled and exhaled repeatedly. Therefore, the accumulating carbon dioxide in the air must be removed from the breathing circuit. Normal breathing air contains 21% oxygen. During one breath, approximately 4% of the oxygen is removed from the inhaled air and replaced by a corresponding amount of exhaled carbon dioxide (CO₂). In principle, a certain volume of air can be “breathed through” several times until its oxygen content is exhausted, but the exhaled carbon dioxide accumulates in the air. There are also physiological risks associated with too much carbon dioxide in the inhaled air: from 5% onwards it leads to unconsciousness, and from 8% onwards over a longer period it leads to death.

In this regard, the device according to the invention has a carrier element in which a CO₂ absorber is arranged. The CO₂ absorber is consumed during hypoxia treatment and/or hypoxia training and therefore usually needs to be replaced before or after each hypoxia treatment and/or hypoxia training or after consumption. The carrier element contains the CO₂ absorber and is arranged in the breathing circuit of the device according to the invention. The CO₂ absorber is usually a mixture of calcium hydroxide and sodium hydroxide in solid form (so-called soda lime). During hypoxia treatment and/or hypoxia training, the air flows through the soda lime, in which the carbon dioxide is first bound to sodium hydroxide, which is then regenerated by the calcium hydroxide, also known as slaked lime, which is also contained therein.

In a refinement of the invention, the CO₂ absorber is arranged in a container, wherein the container is arranged in the carrier element. The container holds the powdered CO₂ absorber, wherein the container is embodied as an exchangeable cartridge that can be replaced with an unused cartridge before or after the hypoxia treatment and/or hypoxia training.

In a further configuration of the invention, the carrier element has an opening through which the container can be inserted into the carrier element. The opening allows the container to be replaced before or after hypoxia treatment and/or hypoxia training.

In a further embodiment of the invention, the carrier element can be closed. The opening for replacing the container can be closed and, in particular, reopened.

In a refinement of the invention, the carrier element has a body and a flap, wherein the carrier element can be closed with the flap. The flap is arranged so as to be movable relative to the carrier element and opens or closes the carrier element.

In a further configuration of the invention, a seal is arranged between the flap and the body of the carrier element, which seal is suitable for closing the carrier element airtightly with the flap. The CO₂ absorber arranged in the container therefore only absorbs carbon dioxide from the user's breathing air and is not contaminated by carbon dioxide from the ambient air.

In a further configuration of the invention, the container can be connected to the supply line and/or the discharge line in such a way that the gas mixture for performing the hypoxia treatment and/or the hypoxia training can be routed through the container. This ensures that the CO₂ absorber arranged in the container removes the carbon dioxide from the gas mixture.

In a further aspect of the invention, a seal is arranged between the container and the supply and/or discharge line, which seal prevents the gas mixture from escaping from the container and the supply and/or discharge line. The CO₂ absorber arranged in the container therefore only absorbs carbon dioxide from the user's breathing air and is not contaminated by carbon dioxide from the ambient air.

In a further configuration of the invention, the container is arranged in the supply and/or discharge line in such a way that the gas mixture is forced through the container. The container and the CO₂ absorber arranged therein are therefore exposed to the breathing air of a user. The carbon dioxide in the gas mixture is effectively absorbed.

In a further embodiment of the invention, the device has a supply line and a discharge line. Separate supply and discharge lines ensure that a user only inhales air freed from carbon dioxide, making the device according to the invention particularly safe to use.

The object mentioned above is furthermore achieved by means of the method for performing hypoxia training according to claim 11. Further advantageous configurations of the invention are also set out in the dependent claims.

The method according to the invention for performing hypoxia training has two method steps: In the first method step, a CO₂ absorber is inserted into a carrier element of a device for providing a hypoxic gas mixture. The carrier element is arranged in the breathing circuit of the device for providing a hypoxic gas mixture and is suitable for receiving a CO₂ absorber.

In the second step, the gas delivery device is connected to the mouth and/or nose of the user. The gas delivery device is usually embodied as a breathing mask that a user wears over the breathing openings (mouth and nose) when performing hypoxia treatment and/or hypoxia training. This ensures that the user breathes only the hypoxic gas mixture that the device for providing a hypoxic gas mixture provides and feeds into the breathing circuit.

In a refinement of the invention, the CO₂ absorber is arranged in a container, wherein the container is arranged in the carrier element. The container holds the powdered CO₂ absorber, wherein the container is embodied as an exchangeable cartridge that is replaced with an unused cartridge before or after the hypoxia treatment and/or hypoxia training.

In a further configuration of the invention, the container is connected to a supply and/or discharge line of the device for providing a hypoxic gas mixture. The supply line is suitable and intended to conduct a gas mixture from a gas reservoir to the gas delivery device. The discharge line is suitable and intended to conduct a gas mixture from a gas delivery device to a gas reservoir. The supply and/or discharge line is usually embodied as a flexible hose and connects the gas delivery device to the gas reservoir in a gas-tight manner. The container is connected to the supply line and/or the discharge line in such a way that the gas mixture is routed through the container for performing the hypoxia treatment and/or hypoxia training. This ensures that the CO₂ absorber arranged in the container removes the carbon dioxide from the gas mixture.

In a further embodiment of the invention, when connecting the container to a supply and/or discharge line of the device for providing a hypoxic gas mixture, an airtight connection is established between the container and the supply and/or discharge line. This prevents the gas mixture from escaping from the container and the supply and/or discharge line. The CO₂ absorber arranged in the container therefore only absorbs carbon dioxide from the user's breathing air and is not contaminated by carbon dioxide from the ambient air.

In a further design of the invention, the container is sealed airtightly when inserted into the device for providing a hypoxic gas mixture. When in the delivery state, the container is sealed in such a way that the CO₂ absorber arranged in the container does not absorb CO₂ from the ambient air and is therefore not already consumed before being inserted into the device for providing a hypoxic gas mixture.

In a refinement of the invention, the container is opened at a first position. In a further aspect of the invention, the container is opened at a second position. In a further configuration of the invention, the first and second positions are arranged opposite one another on the container. Opening the container opens an opening through which the gas mixture flows for performing hypoxia training.

In a further configuration of the invention, the container connects a first section of the supply and/or discharge line to a second section of the supply and/or discharge line. In a further embodiment of the invention, the first and/or second position is connected to the connection section of the first and/or second section of the supply and/or discharge line. Therefore, the container is arranged in the supply and/or discharge line and the CO₂ absorber arranged in the container therefore only absorbs carbon dioxide from the user's breathing air and is not contaminated by carbon dioxide from the ambient air.

The stated object is further achieved by means of the container with a CO₂ absorber for performing hypoxia training according to claim 22.

The container with a CO₂ absorber for performing hypoxia training is sealed according to the invention. In particular, when in the delivery state the container is sealed airtightly. When in the delivery state, the container is sealed in such a way that the CO₂ absorber arranged in the container does not absorb CO₂ from the ambient air and is therefore not already consumed before being inserted into the device for providing a hypoxic gas mixture.

In a refinement of the invention, the container has one or more opening areas, wherein the opening areas are suitable and intended for opening the container in the opening areas. In a further aspect of the invention, the opening area has an opening. Opening the container opens the openings arranged in the opening areas through which the gas mixture flows for performing hypoxia training. The container is embodied to be airtight to the ambient air.

In a further configuration of the invention, the opening is sealed airtightly when in the delivery state. When in the delivery state, the openings of the container are sealed in such a way that the CO₂ absorber arranged in the container does not absorb CO₂ from the ambient air and is therefore not already consumed before being inserted into the device for providing a hypoxic gas mixture.

In a further embodiment of the invention, the opening area of the container has a sealing surface. In a further development, the sealing surface has a seal. The seal prevents the gas mixture for performing hypoxia training from escaping from the container and the supply and/or discharge line, while at the same time preventing the CO₂ absorber arranged in the container from being contaminated by carbon dioxide from the ambient air.

Exemplary embodiments of the device according to the invention for providing a hypoxic gas mixture, the method according to the invention for performing hypoxia training, and the container according to the invention with a CO₂ absorber for performing hypoxia training are shown in simplified schematic form in the drawings and will be explained in more detail in the following description.

In the drawings:

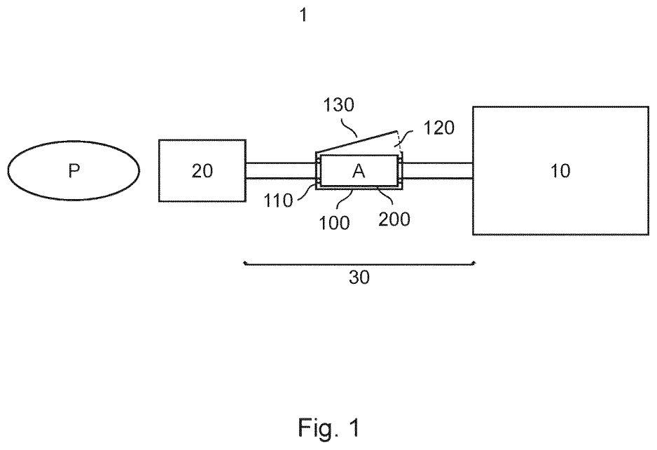

FIG. 1: Device according to the invention for providing a hypoxic gas mixture, a supply and discharge line

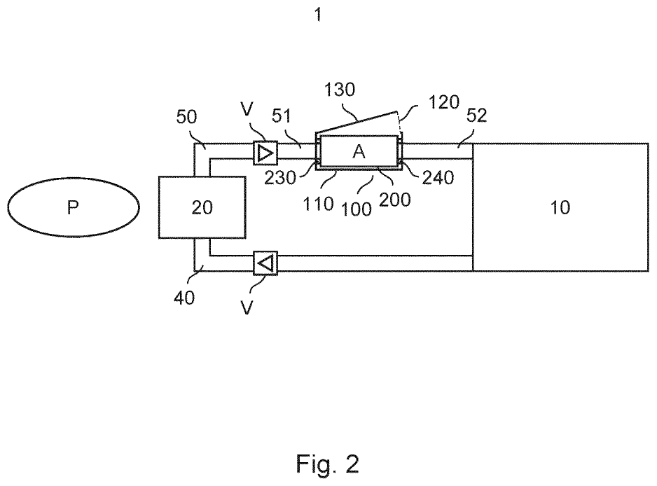

FIG. 2: Device according to the invention for providing a hypoxic gas mixture, supply and discharge lines separated, carrier element in discharge line

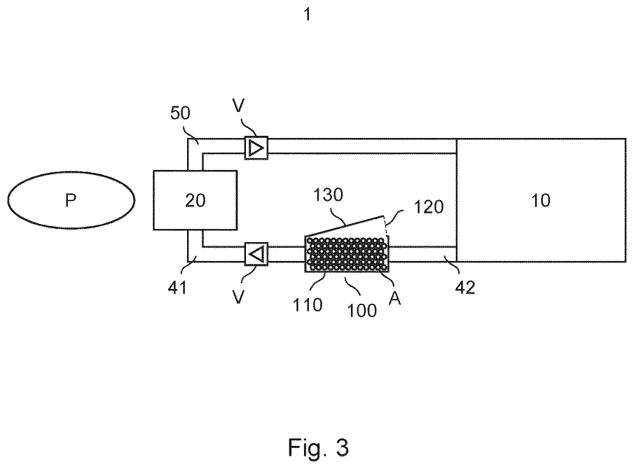

FIG. 3: Device according to the invention for providing a hypoxic gas mixture, supply and discharge lines separated, carrier element in supply line

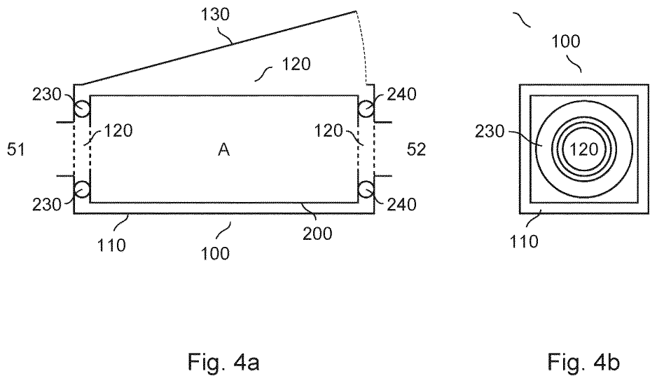

FIG. 4 a: Side view of a carrier element

FIG. 4 b: Front view of a carrier element

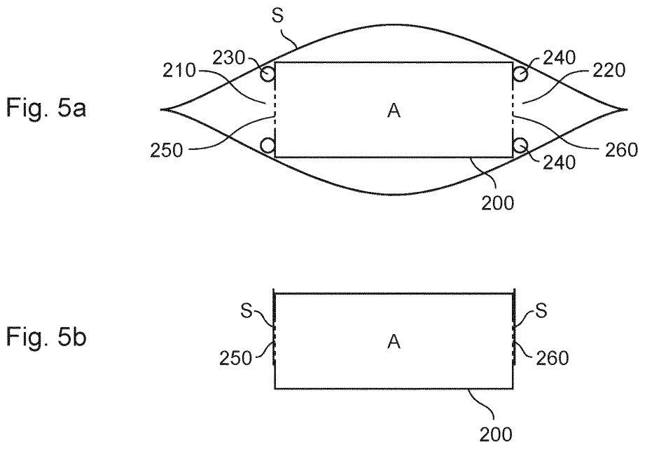

FIG. 5 a: View of a sealed container

FIG. 5 b: Another view of a sealed container

FIG. 1 shows a view of a device 1 for performing hypoxia treatment and/or hypoxia training. Device 1 has gas delivery device 20, which is embodied as a breathing mask and is worn by the user P over the breathing openings (mouth and nose) during the hypoxia treatment and/or hypoxia training. Device 1 also has gas reservoir 10. Gas reservoir 10 and gas delivery device 20 are connected to each other in a gas-tight manner via flexible gas line 30. Gas line 30 has carrier element 100 for receiving CO₂ absorber A. In all exemplary embodiments shown here, CO₂ absorber A is a mixture of calcium hydroxide Ca(OH)₂ and sodium hydroxide NaOH, also referred to as soda lime. Carrier element 100 is arranged in gas line 30 in such a way that the gas mixture is routed through carrier element 100 during the hypoxia treatment and/or hypoxia training. Carrier element 100 has body 110 and opening 120. Opening 120 can be opened and closed by means of flap 130.

A preferred embodiment of device 1 according to the invention for performing hypoxia treatment and/or hypoxia training is shown in FIG. 2. Device 1 also has gas delivery device 20. Gas delivery device 20 is connected to gas reservoir 10 via a flexible supply line 40 and a likewise flexible discharge line 50. Supply line 40 and discharge line 50 each have a one-way valve V. One-way valves V ensure that user P only inhales the air from supply line 40 and thus prevent user P from inhaling CO₂-containing air from discharge line 50 when inhaling.

In this exemplary embodiment, discharge line 50 has carrier element 100 for receiving CO₂ absorber A. Carrier element 100 has body 110 and opening 120, which can be opened and closed airtightly by means of flap 130. Flap 130 has a suitable seal 140 for this purpose. Discharge line 50 has first section 51 and second section 52, which are connected airtightly to each other by carrier element 100.

CO₂ absorber A is arranged in a container 200 (see FIG. 4, FIG. 5). For performing the hypoxia treatment and/or hypoxia training, container 200 is inserted into carrier element 100 with flap 130 open before the start of the hypoxia treatment and/or hypoxia training. Carrier element 100 is closed airtightly by means of flap 130 and gas delivery device 20 (breathing mask) is connected airtightly to the mouth and nose of user P.

After that, the actual hypoxia treatment and/or hypoxia training begins, wherein user P inhales and exhales the same air repeatedly. CO₂ absorber A prevents suffocation. For this purpose, device 1 has measuring apparatuses that permanently monitor the health status of user P during the hypoxia treatment and/or hypoxia training. In particular, a pulse oximeter is used to monitor the heart rate and the oxygen content of the blood. The measuring apparatuses are connected to a control unit that sends out an alarm signal in the event of complications, e.g. low oxygen levels in the user's blood, so that the hypoxia treatment and/or hypoxia training is immediately stopped.

FIG. 3 shows a further exemplary embodiment of device 1 according to the invention for performing hypoxia treatment and/or hypoxia training. Device 1 also has gas discharge device 20. Gas delivery device 20 is connected to gas reservoir 10 via a flexible supply line 40 and a likewise flexible discharge line 50. Supply line 40 and discharge line 50 each have a one-way valve V. In this exemplary embodiment, carrier element 100 with body 110, flap 130 and opening 120 is arranged in supply line 40, wherein first section 41 of supply line 40 and second section 42 of supply line 40 are each connected airtightly to carrier element 100.

In this exemplary embodiment, CO₂ absorber A is not arranged in a container 200, but is arranged as powdered granules in carrier element 100 in such a way that when performing hypoxia treatment and/or hypoxia training, the breathing air is forced through CO₂ absorber A.

An exemplary embodiment of a carrier element 100 provided with container 200 in the delivery state is shown in FIG. 4. Carrier element 100 has a cuboid shape (FIG. 4 a), which is formed by body 110 for receiving container 200. By means of flap 130, body 110 can be opened or closed airtightly. For this purpose, a seal 140 is arranged between flap 130 and carrier element 100 or body 110. Opening 120 is dimensioned such that container 200 can be inserted into body 110 and removed from it.

On the opposite end faces (FIG. 4 b), carrier element 100 is connected airtightly on one side to first section 41, 51 of supply line 40 or discharge line 50, and on the opposite side to second section 42, 52 of supply line 40 or discharge line 50. For this purpose, carrier element 100 has a circular seal 230, 240 on the inner sides of the end faces, and container 200 has correspondingly embodied sealing surfaces on the outer sides of the end faces in each case. Seals 230, 240 can alternatively also be arranged on container 200 (see FIG. 5 a), carrier element 100 then does not require such seals 230, 240. The gas mixture for performing hypoxia treatment and/or hypoxia training is therefore forced through container 200.

FIG. 5 shows exemplary embodiments of containers 200 in the delivery state, which contain CO₂ absorber A. CO₂ absorber A is soda lime, a mixture of calcium hydroxide Ca(OH)₂ and sodium hydroxide NaOH. In this exemplary embodiment, container 200 is cylindrical and has an opening area 210, 220 on each of the end faces, at which container 200 can be opened. Each of the opening areas 210, 220 has an opening 250, 260.

Container 200 can have sealing surfaces on the end faces, each with a circular seal 230, 240 (FIG. 5 a). In this case, carrier element 100 itself does not have any corresponding seals to connect container 200 airtightly in such a manner to discharge line 40 or supply line 40. Alternatively, carrier element 100 has seals 230, 240 (see FIG. 4). In this case, container 200 itself has no seals (FIG. 5 b).

In order to prevent CO₂ absorber A from being loaded with CO₂ from the ambient air, container 200 is sealed when in the delivery state. Seal S can be embodied as a plastic film and completely enclose container 200 (FIG. 5 a). To remove container 200, a user P opens seal S and removes it. Alternatively, only opening areas 210, 220 and in particular openings 250, 260 themselves are each provided with a seal S. To insert container 200 into carrier element 100, a user P removes seal S.

LIST OF REFERENCE NUMERALS

| | |
|---|---|
| 1 | Device for providing a hypoxic gas mixture |
| 10 | Gas reservoir |
| 20 | Gas delivery device/ventilation mask |
| 30 | Gas line |
| 40 | Supply line |
| 41 | First section of the supply line |
| 42 | Second section of the supply line |
| 50 | Discharge line |
| 51 | First section of the discharge line |
| 52 | Second section of the discharge line |
| 100 | Carrier element |
| 110 | Body |
| 120 | Opening |
| 130 | Flap |
| 140 | Seal between body and flap |
| 200 | Container |
| 210, 220 | Opening area |
| 230, 240 | Seal between opening area and supply/discharge line |
| 250, 260 | Opening |
| A | CO₂ absorber |
| P | User |
| S | Sealing of the container |
| V | One-way valve |

1. A device (1) for providing a hypoxic gas mixture, having
a gas reservoir (10),
a gas delivery device (20),
a supply line (40) which is suitable and intended to route a gas mixture from the gas reservoir (10) to the gas delivery device (20), and/or a discharge line (50) which is suitable and intended to route a
gas mixture from the gas reservoir (10) to the gas delivery device (20), and
a carrier element,
wherein a CO₂ absorber (A) can be arranged in the carrier element (100).

2. The device (1) for providing a hypoxic gas mixture according to claim 1, characterized in that
the CO₂ absorber (A) can be placed in the carrier element (100) and removed from it.
the CO₂ absorber (A) is arranged in a container (200)
wherein the container (200) can be arranged in the carrier element (100).

3. The device (1) for providing a hypoxic gas mixture according to claim 1,
characterized in that
the carrier element (100) has an opening (120) through which the container (200) can be inserted into the carrier element (100).

4. The device (1) for providing a hypoxic gas mixture according to claim 1,
characterized in that
the carrier element (100) can be closed.

5. The device (1) for providing a hypoxic gas mixture according to claim 4,
characterized in that
the carrier element (100) has a body (110) and a flap (130), wherein the carrier element (100) can be closed with the flap.

6. The device (1) for providing a hypoxic gas mixture according to claim 5,
characterized in that
a seal (140) is arranged between the flap (130) and the body (110) of the carrier element (100), which seal is suitable for airtightly sealing the carrier element (100) with the flap (130).

7. The device (1) for providing a hypoxic gas mixture according to claim 1,
characterized in that
the container (200) can be connected to the supply line (40) and/or the discharge line (50) in such a way that the gas mixture can be routed through the container (200).

8. The device (1) for providing a hypoxic gas mixture according to claim 7,
characterized in that
a seal (230, 240) is arranged between the container (200) and the supply line (40) and/or the discharge line (50), which seal prevents the gas mixture from escaping from the container (200) and the supply line (40) and/or the discharge line (50).

9. The device (1) for providing a hypoxic gas mixture according to claim 7,
characterized in that
the container (200) is arranged in the supply line (40) and/or discharge line (50) in such a way that the gas mixture is forced through the container (200).

10. The device (1) for providing a hypoxic gas mixture according to claim 1,
characterized in that
the device (1) has a supply line (40) and a discharge line (50).

11. A method for providing a hypoxic gas mixture comprising the method steps of:
inserting a CO₂ absorber (A) into a carrier element (100) of a device (1) for performing training with a hypoxic gas mixture,
and
connecting the gas delivery device (20) to the mouth and/or nose of the user (P).

12. The method for providing a hypoxic gas mixture according to claim 11,
characterized in that
the CO₂ absorber (A) is arranged in a container (200)
wherein the container (200) is arranged in the carrier element (100).

13. The method for providing a hypoxic gas mixture according to claim 12,
characterized in that
the container (200) is connected to a supply line (40) and/or discharge line (50) of the device (1) for performing hypoxia training.

14. The method for providing a hypoxic gas mixture according to claim 12,
characterized in that
when connecting the container (200) to a supply line (40) and/or discharge line (50) of the device (1) for performing hypoxia training, an airtight connection is established between the container (200) and the supply line (40) and/or discharge line (50).

15. The method for providing a hypoxic gas mixture according to claim 12,
characterized in that
the container (200) is airtightly sealed when inserted into the device (1) for performing hypoxia training.

16. The method for providing a hypoxic gas mixture according to claim 12,
characterized in that
the container (200) is opened at a first position.

17. The method for providing a hypoxic gas mixture according to claim 12,
characterized in that
the container (200) is opened at a second position.

18. The method for providing a hypoxic gas mixture according to claim 12,
characterized in that
the first and second positions are arranged opposite one another on the container (200).

19. The method for providing a hypoxic gas mixture according to claim 12,
characterized in that
the container (200) connects a first section (41, 51) of the supply line (40) and/or discharge line (50) to a second section (42, 52) of the supply line (40) and/or discharge line (50).

20. The method for providing a hypoxic gas mixture according to claim 19,
characterized in that
the container (200) airtightly connects the first section (41, 51) of the supply and/or discharge line (40, 50) to the second section (42, 52) of the supply and/or discharge line (40, 50).

21. The method for providing a hypoxic gas mixture according to claim 11,
characterized in that
the first and/or the second position is connected to the connection section of the first and/or second section (41, 42, 51, 52) of the supply and/or discharge line (40, 50).

22. A container (200) with a CO₂ absorber (A) for providing a hypoxic gas mixture,
characterized in that
the container (200) is sealed.

23. The container with a CO₂ absorber (A) for providing a hypoxic gas mixture according to claim 22,
characterized in that
the container (200) has one or more opening areas (210, 220), wherein the opening areas (210, 220) are suitable and intended to open the container (200) in the opening areas (210, 220).

24. The container (200) with a CO₂ absorber (A) for providing a hypoxic gas mixture according to claim 23,
characterized in that
the opening area (210, 220) has an opening (250, 260).

25. The container (200) with a CO₂ absorber (A) for providing a hypoxic gas mixture according to claim 24,
characterized in that
the opening (250, 260) is airtightly sealed when in the delivery state.

26. The container (200) with a CO₂ absorber (A) for providing a hypoxic gas mixture according to claim 23,
characterized in that
the opening area (210, 220) of the container (200) has a sealing surface.

27. The container (200) with a CO₂ absorber (A) for providing a hypoxic gas mixture according to claim 26,
characterized in that
the sealing surface has a seal (230, 240).
